(19) United States
(12) Patent Application Publication
(10) Pub. No.: US 2022/0146425 A1
(43) Pub. Date: May 12, 2022

(54) IMPROVEMENTS IN OR RELATING TO AN OPTICAL ELEMENT

(71) Applicant: Vidya Holdings Ltd, London (GB)

(72) Inventors: Stefan Leo Van Workum, London (GB); Callum Robertson Smith, London (GB); Marko Dorrestijn, London (GB); David R. Klug, London (GB)

(73) Assignee: Vidya Holdings Ltd, London (GB)

(21) Appl. No.: 17/435,372

(22) PCT Filed: Feb. 28, 2020
(86) PCT No.: PCT/GB2020/050486

§ 371 Date: Aug. 31, 2021

(30) Foreign Application Priority Data
Mar. 1, 2019 (GB) 1902788.7
Mar. 1, 2019 (GB) 1902789.5

(51) Int. Cl. G01N 21/64; G01N 35/00; G01N 33/68

(52) U.S. Cl. CPC G01N 21/648; G01N 35/00732; G01N 33/6803; G01N 2035/00782; G01N 2035/00752; G01N 2035/00861; G01N 2021/6478

(57) ABSTRACT

An assay cartridge for detecting a target component in a fluid is provided. The assay cartridge including an optical element comprising: a light pathway comprising an input surface, reflective surface and output surface configured to enable light to enter, reflect and create an evanescent field in the vicinity of the reflective surface and exit the element; a plurality of capture components deposited on the reflective surface in the vicinity of the evanescent field; and a transmission surface configured to enable emissions from the evanescent field to exit the element; wherein the assay cartridge is a single use cartridge.

The present invention relates to improvements in or relating to an optical element for use in a cartridge and a method for fabricating a plurality of optical elements.

Analytic or diagnostic systems often include a disposable cartridge and a detector. A test cartridge comprising an optical element can be used for tracking the level of key analytes or biomarkers which may be taken as a measure of wellness or conversely, to identify the presence or absence of specific biomarkers indicative of disease states. Alternatively there may be interest in levels of contaminants in other materials such as water. Optical elements form part of this test cartridge and can be provided with capture components such as antibodies on at least one surface in order to bind to its complementary target components within a sample e.g. a bio-fluid such as urine, saliva, tears, sweat or blood. Assays are often performed on a device such as an assay cartridge in order to detect target components within a sample which is within the cartridge. If a sandwich assay format is used with emissively labelled additional antibodies not bound to the surface, then the target component can then be detected using total internal reflection (TIR) and more particularly total internal reflection fluorescence (TIRF).

TIRF exploits a thin light field extending into the area of the cartridge containing the sample, on the order of a few hundred nanometres thick, which is generated by total internal reflection. Total internal reflection occurs at the interface between a higher refractive-index medium and a lower refractive-index medium. Above a critical angle, defined by the refractive indices of the two media, light travelling in a higher refractive-index medium incident on a lower refractive-index medium is totally reflected. This total internal reflection generates an exponentially decaying light field, known as an evanescent wave. The evanescent wave can be used to excite luminescent molecules in very close proximity to the boundary between the two media. These luminescent molecules will consequently emit light at a certain wavelength, which can be selectively detected to provide information on the boundary region. The luminescence may arise from fluorescence or phosphorescence.

The through-objective configuration is a well-established technique for achieving total internal reflection fluorescence. However, the use of a through-objective configuration requires the use of an index-matching material to interface the sample with a high numerical aperture microscope objective. This is highly undesirable for deskilled instruments due to the complexity involved with applying and removing index-matching materials in a reproducible manner between each cartridge. It can also be a complication when high throughput measurements are required as the index matching fluid has to be cleaned and replaced regularly. Furthermore, the through-objective technique has a limited field of view and therefore often requires interrogating each deposited target component such as an antibody spot individually, thus this would require the need for moving parts to allow the system to move between each spot.

Waveguiding is another technique that can be used for TIR measurements. Waveguiding removes the requirement for an index-matching material. However, the signal to noise ratio achieved by waveguiding is dramatically reduced compared to the TIRF measurements performed using the through-objective technique. This is largely because multimode waveguides allow light propagating over a range of angles to impinge on the boundary surface, increasing the net depth of the evanescent field and also potentially allowing light below the critical angle to be transmitted.

A prism-based system provides a higher signal to noise ratio compared with the waveguiding approach during TIRF measurements of the cartridge. However, the prism-based system would require interfacing a prism with a coverslip or a microscope slide or other optical element using an index-matching material so as to allow the evanescent wave to be launched into the boundary between the optical element and the medium containing the sample within the cartridge. This would be undesirable due to the complexity of applying and removing a liquid index-matching material between each cartridge. Additionally, moving parts can be associated with a higher failure rate. Furthermore, the use of index-matching material increases the risk of contamination such as bubbles or unwanted particulates, which can scatter light and thus contribute to noise during the measurement reducing the reliability of the measurement. Alternatively, proprietary solid phase index matching solutions are known, but use of this approach would also push up manufacturing costs of making the cartridge with a prism-based system.

Fabricating optical elements typically involves cutting an optical element into appropriate dimensions which requires precise manufacturing machinery. In addition, the surfaces of the optical elements are often optically polished and finely ground during the manufacturing process. These processing steps can often be time and resourcefully intensive. Hence, the costs required for fabricating optical elements can be substantial and does not allow for major cost savings when fabricated in large numbers as these processes have to be applied to each optical element. Thus, fabricating cheap optical elements in large numbers provides an enormous challenge for manufacturers and users.

Therefore, it would be extremely beneficial to identify and/or develop a high-volume fabrication process to manufacture the optical element within the test cartridge. It is therefore an objective to reduce the costs of fabricating the optical elements in large numbers.

It is against this background that the present invention has arisen.

According to the present invention there is provided an optical element comprising:
a light pathway comprising an input surface, reflective surface and output surface configured to enable light to enter, reflect and exit the element; wherein capture components are deposited on the reflective surface; and wherein the element further comprises a transmission surface configured to enable emissions from the capture components to exit the element.

The capture components may be deposited via a printing process involving a liquid suspension containing the capture component being printed directly onto a predetermined area of the reflective surface. In some embodiments a pattern of spots is printed. Each spot is printed as a droplet containing capture components such as antibodies. The droplets can be left on the surface for a period of time to allow physisorption of the antibodies to the surface. Any excess antibodies can then be washed off to leave an approximate “monolayer” of antibodies.

Alternatively or additionally, a relevant part of the reflective surface or even the whole reflective surface may be functionalised by the provision of suitable primers and the capture components may be flowed over the surface or deposited in a localised way. The functionalisation of the surface can be achieved by a surface treatment process or by depositing the reactive components directly on the reflective surface. The capture components can then be flowed over the reflective surface or deposited over the reactive areas.

The provision of the capture components directly onto the reflective surface of the prism negates the need for index-matching materials. This can be advantageous as it may reduce the complexity and variability associated with using liquid index-matching material and the cost and variability of using a solid index matching solution.

In use a sample containing a target component is introduced into the cartridge. The sample comes into contact with detection reagents and capture components, either simultaneously or sequentially. Binding between the detection reagent, target component and capture component occurs to form a sandwich assay. As a result of the attachment of the capture component on the reflective surface of the optical element, the sandwich assay is localised on this surface. As a result of either the provision of a label attached to the detection reagent, or the inherent luminescent qualities of the detection reagent, light will be emitted from the sandwich assay when it is excited by incident light on the light pathway. Because the excitation occurs only within the evanescent field that develops in the vicinity of the reflective surface, only luminescence will only occur in sandwich assays localised on the surface. Detection reagents, or their labels which are not so localised will not receive this excitation and will not therefore luminesce.

In some embodiments, the input surface and/or the output surface may be a refractive or a diffractive or a transmissive surface.

The refractive or diffractive surfaces may direct the light such as an incident light beam to the reflective surface. The selection of refractive, diffractive or transmissive surface may allow the light pathway to be optimally incorporated into the device into which the optical element is provided.

In some embodiments, the emissions from the labels may be luminescence. In these cases the luminescence may be generated by a label attached to the detection reagents or originate from the detection reagent itself. The luminescence emitted by the detection reagents may be fluorescence or phosphorescence. Fluorescence is a common method for detecting labelled samples in biological systems, via TIRF microscopy, due to its outstanding signal-to-noise and signal to background ratios. Due to the low penetration depth of the evanescent field, which forms just in the vicinity of the reflective surface of the optical element, the out-of-focus excitation of luminophores that are not on the surface is minimized and therefore hardly any background fluorescence occurs.

In some embodiments the emission from the detection reagents or their labels may be in the form of scattering, such as Mie, Rayleigh or Raman scattering. In these cases highly scattering labels may be attached to the detection reagents or the scatter may originate from them directly.

In some embodiments, the light may undergo a single reflection at the reflective surface. The incident light beam could reach the target region for a single reflection through an angular change resulting from refraction of the incident light beam within the optical element.

Alternatively, the incident light beam could reach the target region for a single reflection without an angular change resulting from refraction.

In some embodiments, the light may undergo a plurality of reflections at the reflective surface. The geometry and refractive index of the material from which the optical element is formed may enable incident light to make a small, discrete number of reflections on the reflective surface.

Alternatively, the optical element may be configured as a waveguide so that an optical field develops within the element with the light filling the element and making an effectively indistinguishable number of reflections via each surface due to the waveguiding.

In some embodiments, the optical element can be a prism, or a dove prism, or a cuboid. In some embodiments, the optical element may be triangular. The angle of input and output surface can be set to Brewster's angle, which when combined with an appropriately polarised input light source can result in drastically reduced unwanted reflections.

The input light beam can be incident on the optical element at any angle that will achieve total internal reflection. This range of angles can be determined for any optical element by applying Snell's law for the material from which the optical element is fabricated. The input light beam may be configured to be incident perpendicular to the input surface, in which case it will pass straight through. Alternatively, the input beam may be incident non-perpendicular to the input surface and it will therefore refract at the input surface and then progress to the reflective surface at which point, it must be incident at an angle that facilitates total internal reflection.

The angle between the input surface and the reflective surface is selected to optimise the light pathway, through the optical element. The angle may be acute, i.e. less than 90° or it may be 90°. In the later case, the prism may be referred to as a cuboid. The angle between the reflective surface and the output surface is selected to comply with the packaging requirements of the optical element. It may be the same angle as that between the input surface and the reflective surface. This may be advantageous because it simplifies the assembly of the device as the optical element can be inserted with either of the bases as the input surface. However, if the packaging of the element dictates, an asymmetric configuration may be utilised.

In some embodiments, the transmission surface can be a diffractive, or a refractive or a non-planar surface. The non-planar surface can be utilised to aid with light collection or imaging application. This is particularly useful for optical elements constructed from polymer, since complex volumes can be fabricated with much greater ease compared with optical glasses.

In some embodiments, the input and/or output surfaces may further comprise at least one diffractive grating, which may be transmissive or reflecting. Gratings at the input and/or output surfaces of the optical element may be utilised for splitting the light into several beams travelling in different directions.

The capture component may be an antibody. Alternatively or additionally, the capture component could be a nucleic acid such as DNA, RNA, mRNA or microRNA, or chemically modified nucleic acid; it could be a protein, or a modified protein; it could be a hormone; or a tethered drug configured to capture a protein. There may be a single capture component provided. Alternatively, a combination of multiple capture components may be provided. It is advantageous to combine multiple capture components since this allows a more powerful diagnostic ability. In some embodiments, the multiple capture components deposited on the reflective surface may be spatially addressable.

In another aspect of the invention, there is provided an assay cartridge for detecting a target component in a biological fluid, the assay cartridge comprising an optical element according to the previous aspect of the invention; wherein the capture component is selected to capture the target component to be detected.

In some embodiments, the optical element may be disposable. In this context disposable is intended to include single, or low number of uses, including where a number of readings are taken over time for a single sample of biological fluid and the element is then disposed of. The element may also be recyclable, but it must be removed and reconditioned before being used again. This approach ensures that the risk of cross contamination between samples is completely eliminated as the cartridge is used for a single sample only. By ensuring that the element is recyclable, responsible use of resource is ensured.

The rationale for single use cartridges is that the cartridges are intended for use in a non-controlled environment, for example at the point of care, in the home, or in the field. The cartridges are therefore configured to be single use to avoid degradation of performance.

In some embodiments, the single use nature of the optical element may be assured by the presence of an irreversible spot. This spot can be configured to light up regardless of the nature of the sample to note that a sample has been flowed across the optical element and therefore the cartridge has been used. If the same cartridge is used again, this spot will be lit up from the outset which will clearly evidence that the cartridge has been used more than once and therefore the results should be regarded as suspect.

The spot could be streptavidin and the cartridge could be configured to carry a conjugate of a biotin and a fluorophore over the cartridge with the sample flow. The binding between streptavidin and biotin is one of the strongest non-covalent interactions and therefore, once the interaction has occurred, it can't be reversed by washing or other processing. An initial check of the read-out, prior to the assay being completed, will therefore identify whether the cartridge has been used again. If it has, the results will be invalid.

Conversely, the irreversible spot could be bleached at the end of the assay so that it cannot be activated through a second or subsequent use. In this embodiment, the irreversible spot could still be streptavidin which would definitely light up when a first, legitimate use was made of the cartridge. At the end of the assay, the spot would be irreversibly bleached so that on any subsequent, illegitimate use of the cartridge, the spot would not light up at any stage in the assay. This configuration has the advantage that observation of the cartridge is only required in line with the assay results.

In some embodiments, the irreversible spot may light up in the presence of water, as all of the samples that are intended for use in the cartridge are aqueous. The irreversible spot, in these embodiments, may not be one of the spots in the array, but rather a tape which undergoes a permanent change in the presence of water. The change may be a colour change, for example from white to red. Alternatively or additionally, the change may be a change of transparency so that light can no longer pass through if the cartridge has previously been in contact with water. This would have the advantage of detecting an accidentally water damaged cartridge in addition to a cartridge that has been illegitimately used twice.

In some embodiments, the single use nature of the optical element may be assured by the presence of an identity tag such as a printed barcode or RFID tag. Each identity tag is unique and only one set of data can be accepted and associated with each identity tag. Therefore, if the optical element is reused, any subsequent data submitted to a central database associated with that identity tag will be rejected.

In some embodiments, the single use nature of the optical element may be assured by the provision of a single use clip. In this context a single use clip is a clip that closes once and then cannot be opened again by the user. By physically preventing the cartridge from being opened, the user is prevented from accessing the interior of the cartridge after an initial sample has been provided.

In some embodiments, the biological fluid can be a saliva sample. A saliva sample may be a biological sample as it is a non-invasive procedure. In addition, saliva can be quick and easy to obtain from subjects. Moreover, saliva can contain similar amounts of useful content to blood samples. Alternatively or additionally, the biological fluid may be a blood or a urine sample.

In some embodiments the fluid may be a sample of a fluid such as water from a water supply, stream, lake, sea or ocean.

In some embodiments, the reflective surface may be configured to form a portion of a microfluidic flow channel or a well.

In a further aspect of the invention, there is provided an apparatus for detecting the presence and/or the amount of a target component in a biological fluid, the apparatus comprising
an assay cartridge including an optical element according to a previous aspect of the invention, and
a detector for detecting the presence and/or the amount of the emitted light to provide an indication of the presence and/or the amount of the target component within the sample.

The amount of target component present within a sample may be related to the concentration of the target component within the sample. However, in some embodiments, wherein the sample is a protein, the binding of the protein to the capture component may be imperfect and therefore only a subset of the protein present will be detected. It may be possible to calibrate in order to calculate the actual concentration of the target component present in the sample. In another embodiment the relationship between the detected level of protein and the condition or state of the source of the sample from which the sample was taken can be determined by correlation of the levels with other methods for determining the levels of protein to provide a meaningful readout.

In some embodiments, the optical element further comprises an aperture such as a pinhole. The aperture can be applied to cut out lateral light coming from the sample region.

In some embodiments, the apparatus may further comprise an excitation light source. In some embodiments, the excitation light source may provide an incident light beam which may be configured to generate an evanescent excitation field across the deposited capture component on the reflective surface.

In some embodiments, the detector may further comprise a spatial filter configured to enhance the signal-to-noise ratio of the emitted light. Providing a spatial filter can be advantageous as the spatial filter may be configured to cut out light that does not originate from the sample region.

Furthermore, the spatial filter may be configured to reduce or eliminate out-of-plane fluorescence signal. The use of a spatial filter improves signal to noise ratio of the emitted light and also reduces noise.

In some embodiments, the apparatus may further comprise a first imaging lens positioned between the optical element and the spatial filter. The first imaging lens may be configured to focus the emitted light onto the spatial filter.

In some embodiments, the apparatus may further comprise a second imaging lens positioned between the spatial filter and the detector. The second imaging lens may be configured to focus the emitted light onto the reader.

In some embodiments, the apparatus may further comprise a spectral filter configured to minimise or eliminate one or more scattered or emitted wavelengths.

According to the present invention there is provided a method of fabricating a plurality of optical elements, the method comprising the steps of: heating a preform to a temperature equal to or exceeding the glass transition temperature of the preform; drawing the preform into an elongate strand; and dividing the strand into a plurality of optical elements.

The glass transition temperature can be empirically measured for a given material. For example, silica has a glass transition temperature around 2000° C., whereas some polymers have transition temperatures around 300° C. The preform can be heated to a temperature sufficient to alter its viscosity so that it is appropriate for drawing into an elongate strand. This drawing temperature occurs above the glass transition temperature and below the crystallisation temperature (if present in the material).

This approach offers various advantages. Firstly, a single processed preform can produce thousands of optical elements when drawn into an elongate strand. Moreover, the physical volume of materials required for each unit is considerably reduced and, therefore, the bulk material cost per unit is reduced. Equally significantly, the surfaces produced by the process can be of optical quality, obviating the need for further polishing of surfaces.

In some embodiments, dividing of the strand into a plurality of optical elements may involve cleaving the strand. Cleaving is used herein to refer to any mechanical process for cutting the strand to leave an optical quality surface finish.

In some embodiments, dividing of the strand into a plurality of optical elements may involve a combination of ablation and subsequent polishing. In some embodiments, this processing can be carried out by a CO₂ or other laser, since silica exhibits extremely high absorption at the emission wavelength of a CO₂ lasers and other high power short pulse lasers can be used to ablate glass through multi-photon ionisation

In some embodiments, the step of dividing may occur perpendicular to the direction in which the preform is drawn. This can provide a series of cuboid optical elements.

In some embodiments, the step of dividing may occur at an angle of more than 75° to the direction in which the preform is drawn. An angle of 75° between the direction in which the preform is drawn is the same as “less than 15° ” to the perpendicular direction. This is typically the maximum angle that is possible with commercially available mechanical cleaving methods.

The angle will be selected according to the method of dividing the strand into the plurality of optical elements and also the packaging of the optical element into the cartridge and the constraints that this places on the beam path through the element.

In some embodiments, the cleaving step may take place alternately from each side of the strand so that each optical element has a trapezoidal cross section.

In some embodiments, the two cleaving steps may be provided at substantially the same angle. This will provide a trapezoid with equal length legs. This is preferable where the packaging of the optical element into the cartridge does not force an asymmetric configuration. A symmetrical element can be inserted into the cartridge in either of two possible orientations. It is not possible to insert it the wrong way round as both configurations are equally valid.

However, an asymmetric configuration may provide advantages when the beam pathway through the cartridge is non-trivial and the angles can therefore be selected to achieve an optimal light pathway through the optical element which forms part of the cartridge.

In some embodiments, the preform can be fused silica chosen to be low in impurities. Low impurity fused silica is advantageous because it has low auto fluorescence.

In some embodiments, the dimensions of the strand are dictated by rate at which the strand is drawn. For example, the strand may be drawn from a substantially square cross sectioned preform with side length in the region of 20 mm to50 mm. The height of the preform may be in the range of 20 to 30 cm. The strand may be a 1 mm×1 mm cross section strand, although strands of 2 mm×2 mm or even 3 mm×3 mm could be utilised. The cross section can be kept as small as practically possible to minimise cost of materials. In some embodiments, the cross section of the elongate strand can be 500 μm×500 μm, 400 μm×400 μm or 300 μm×300 μm.

In another aspect of the invention, there is provided a method of fabricating a chip, the method comprising the steps of: fabricating a plurality of optical elements using the method according to a previous aspect of the invention, mounting at least one optical element in a base which in some embodiments may be made from a polymer or polymer mix.

In some embodiments, the mounting step may involve mounting a plurality of optical elements in the polymer base.

In some embodiments, the step of mounting the optical elements may involve mounting the plurality of optical elements parallel to one another.

In some embodiments, the method may further comprise the step of depositing at least one capture component spot onto one of the bases of the optical element.

The depositing of the capture component may occur through a printing process or via the functionalization of part of the surface followed by flowing over a fluid containing the relevant component and then washing off unbound component. The capture component may be one or more of antibodies, nucleic acids such as DNA, RNA, mRNA or microRNA, or one or more modified nucleic acids. Additionally or alternatively, the capture component could be a protein, or a modified protein a hormone, or a tethered drug configured to capture a protein. There may be a single capture component provided. Alternatively, a combination of multiple capture components may be provided. It is advantageous to combine multiple capture components since this allows a more powerful diagnostic ability.

In some embodiments, more than one capture component can be printed onto the base of the optical element.

In some embodiments, the capture components can be printed in a repeating pattern along the base of the optical element.

In some embodiments, the capture components can be printed in a different repeating pattern on each optical element.

The invention will now be further and more particularly described, by way of example only, and with reference to the accompanying drawings, in which:

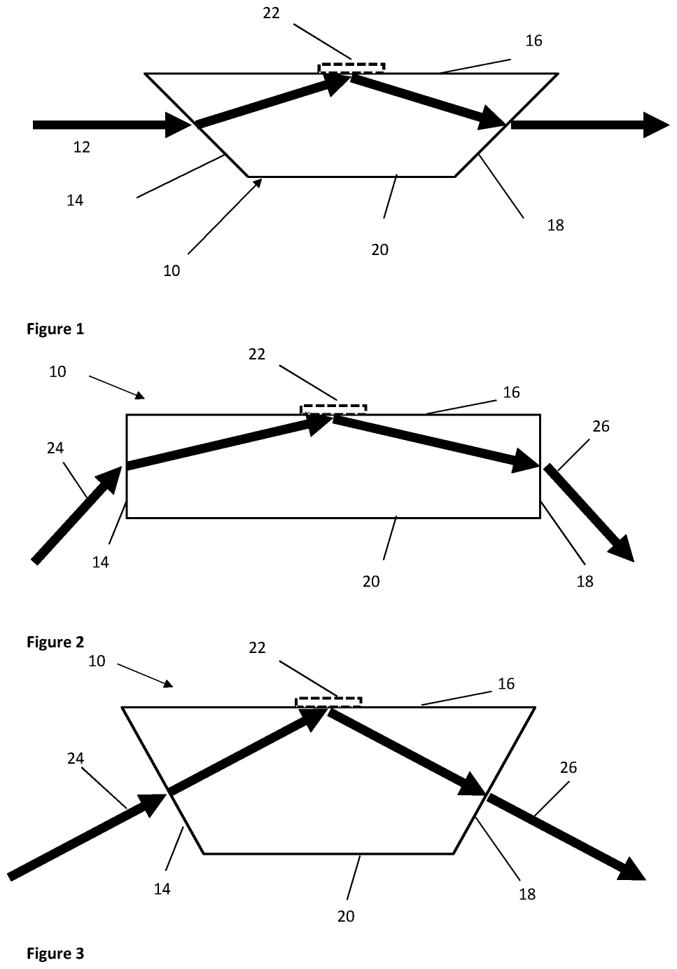

FIG. 1 provides an optical element according to an aspect of the present invention;

FIG. 2 shows a cuboid optical element;

FIG. 3 shows a dove-prism optical element,

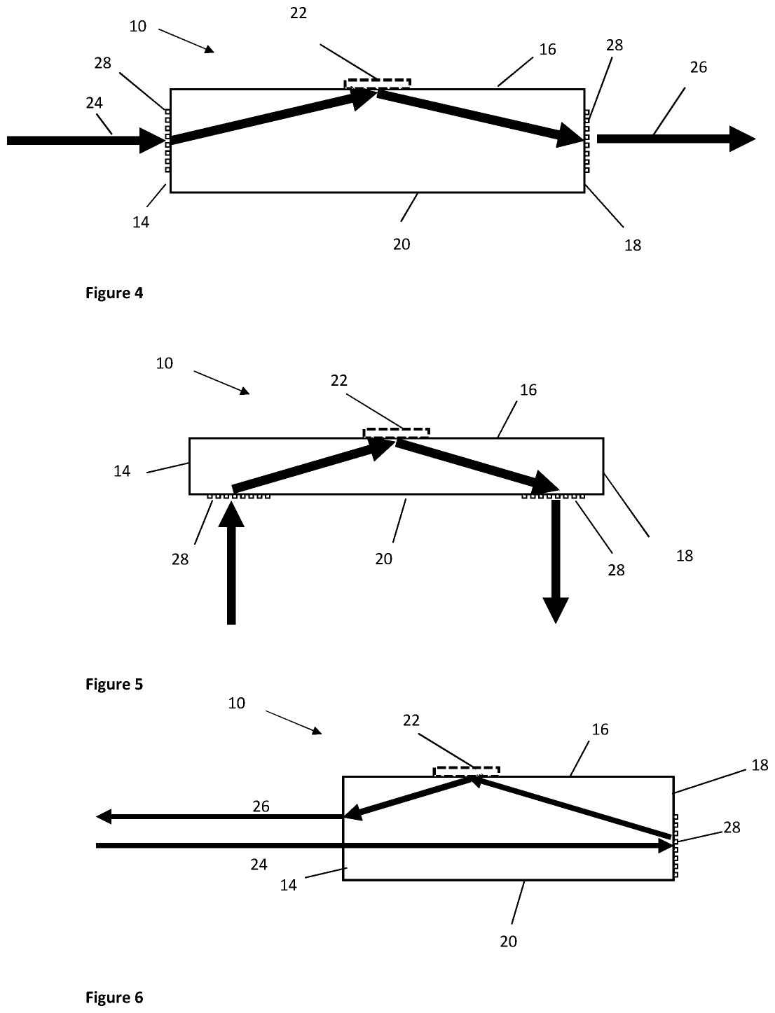

FIG. 4 shows diffraction grating at an input and/or output surface of the optical element according to FIG. 1;

FIG. 5 shows the diffraction grating at a transmission surface of the optical element according to FIG. 1;

FIG. 6 shows the optical element according to FIG. 1 with reflection grating;

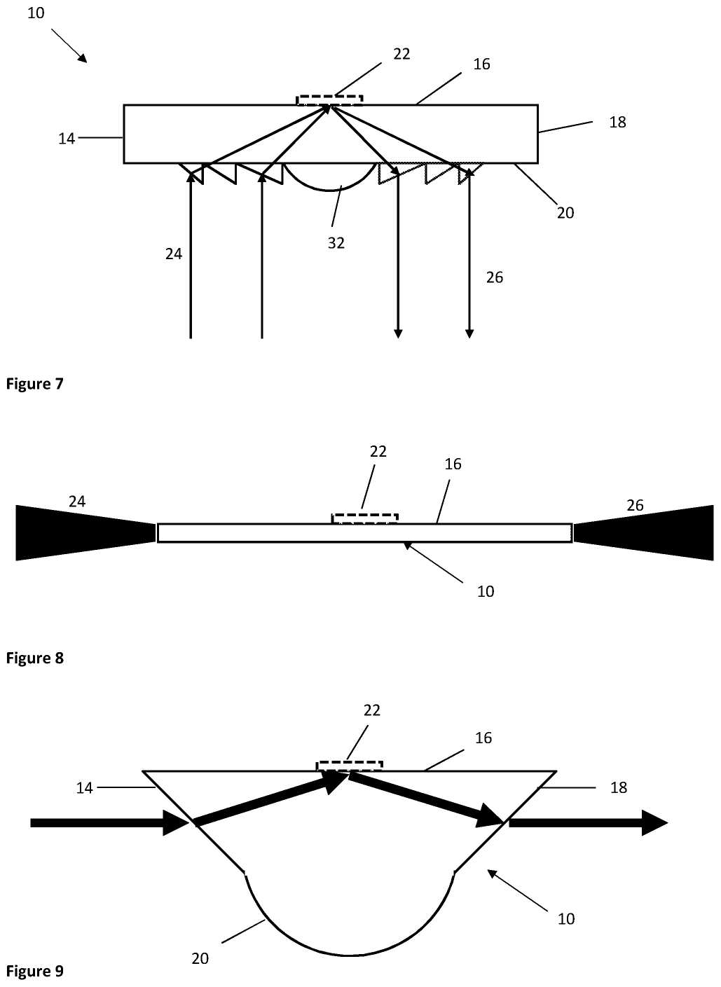

FIG. 7 shows the optical element according to FIG. 1 with a Fresnel lens structure;

FIG. 8 provides an illustration of a waveguiding scenario using the optical element according to FIG. 1;

FIG. 9 shows the optical element according to FIG. 1 with a non-planar transmission surface;

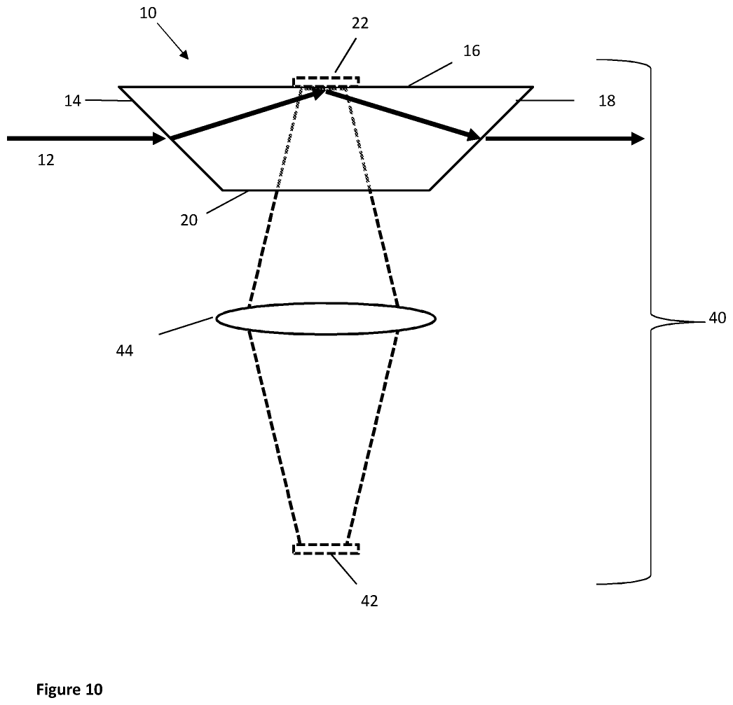

FIG. 10 shows an apparatus set up according to an aspect of the invention;

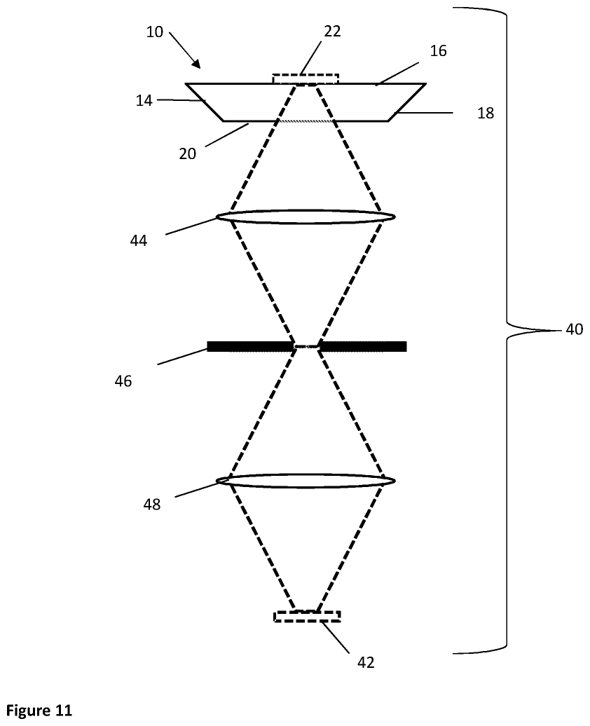

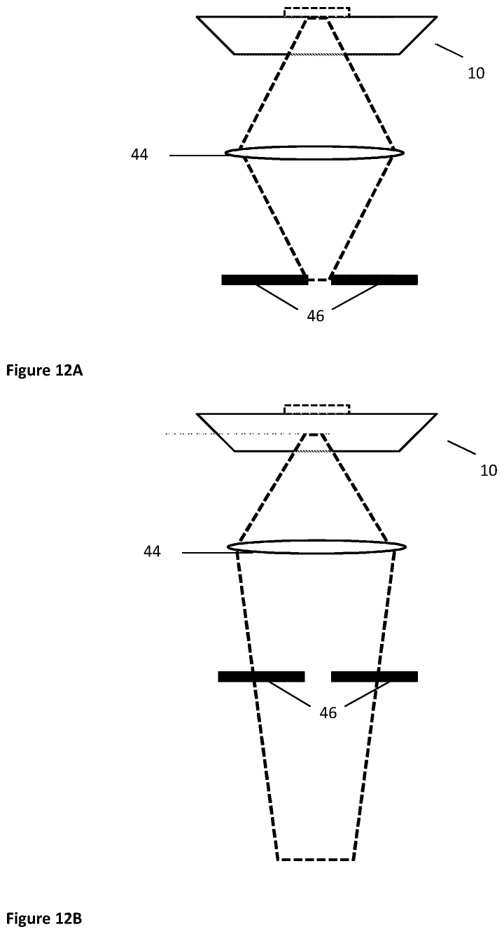

FIG. 11 shows the apparatus according to FIG. 10 with a spatial filter;

FIG. 12A provides an illustration of the spatial filter arrangement;

FIG. 12B provides an alternative illustration of the spatial filter;

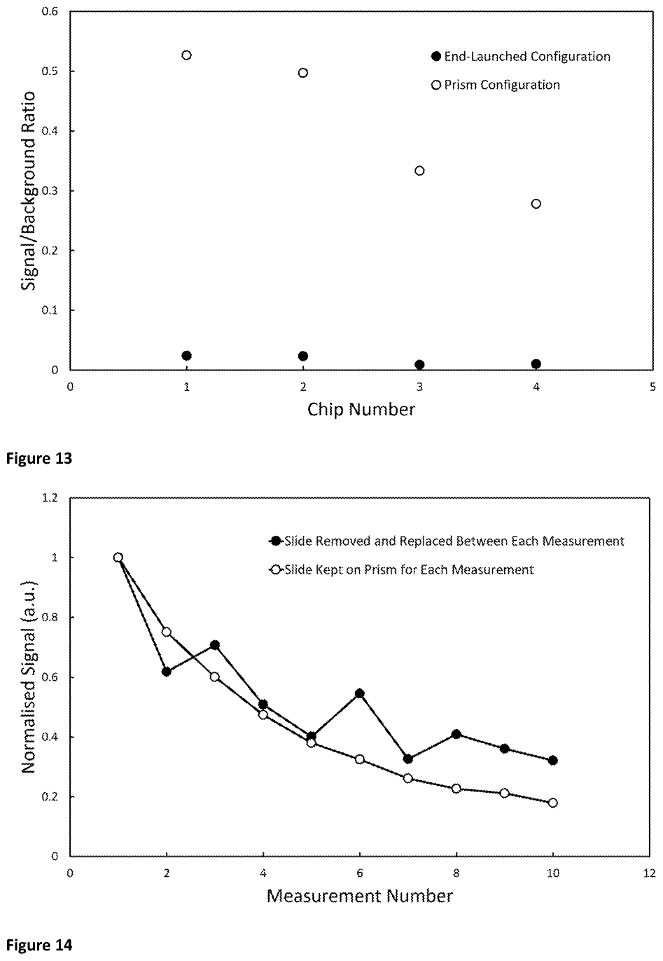

FIG. 13 provides a graph showing the signal to background ratio of the optical element according to FIG. 1; and

FIG. 14 provides a graph showing a comparison between two test cartridges; and

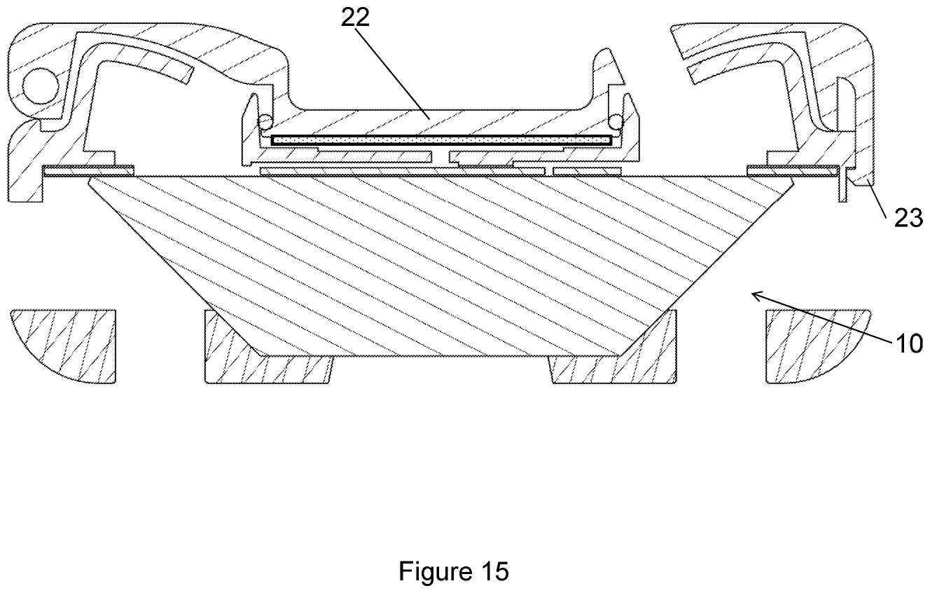

FIG. 15 shows a cross sectional view of a cartridge incorporating to optical element of FIG. 1; and

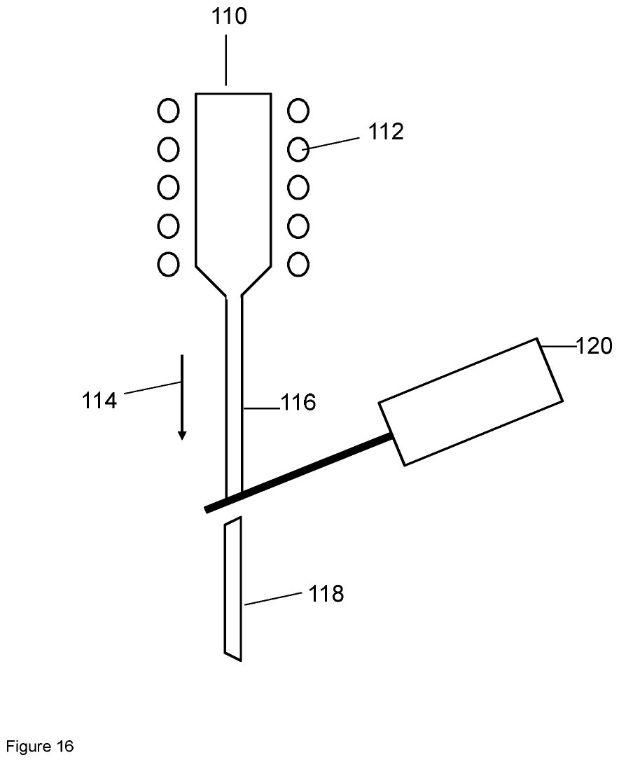

FIG. 16 shows, schematically the steps in the method of fabricating a plurality of optical elements according to the present invention;

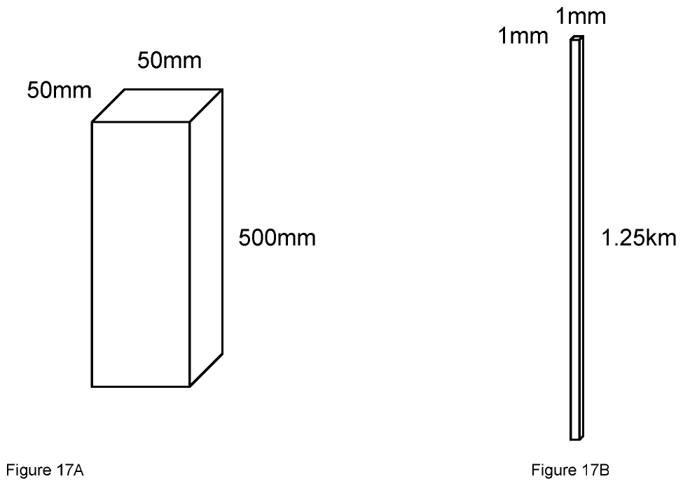

FIG. 17A shows, schematically, a preform prior to the drawing process that forms part of the method illustrated in FIG. 16;

FIG. 17B shows, schematically, an elongate strand following the drawing process that forms part of the method illustrated in FIG. 16;

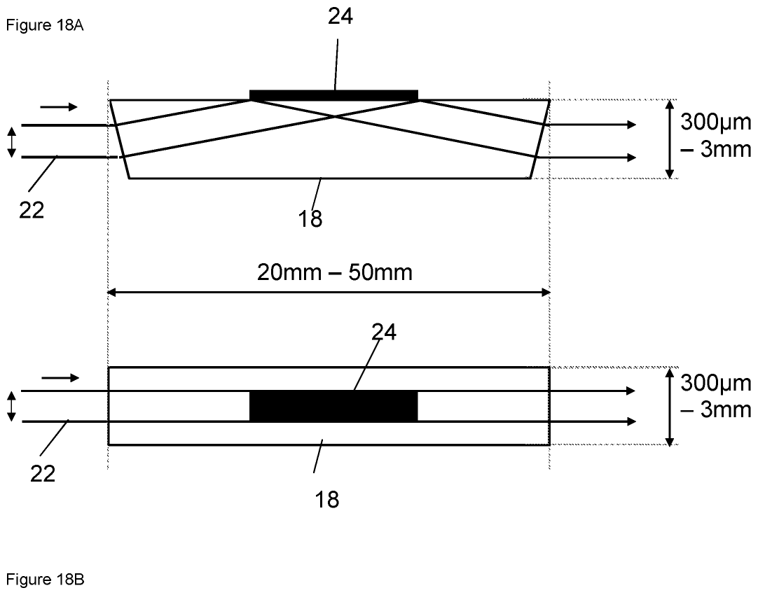

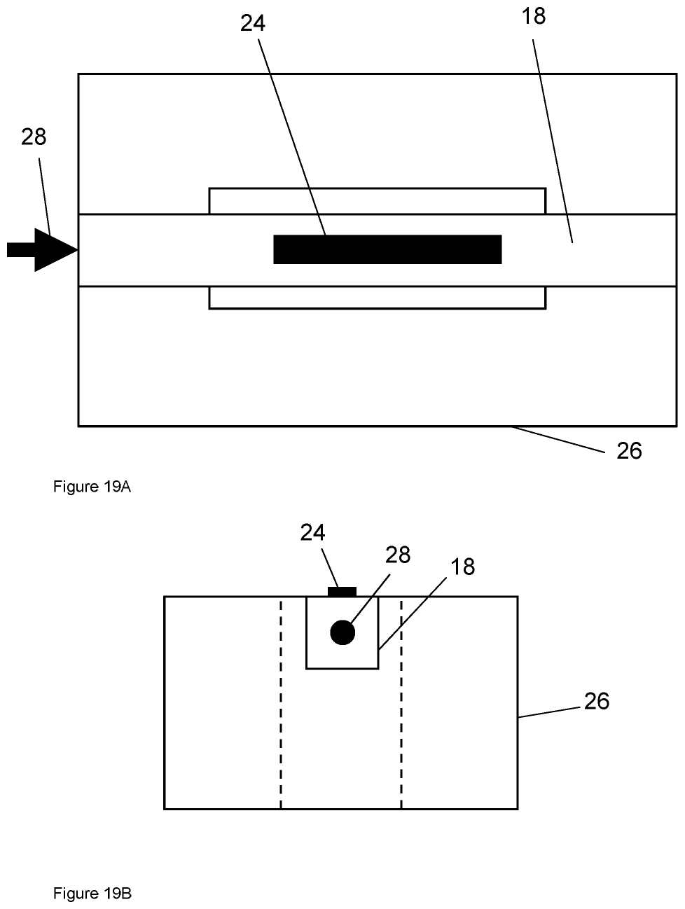

FIGS. 18A and 18B provide, respectively, side and top views of a single optical element fabricated in the method illustrated in FIG. 16;

FIGS. 19A and 19B show, respectively top and side views of a chip containing the optical element illustrated in FIGS. 18A and 18B;

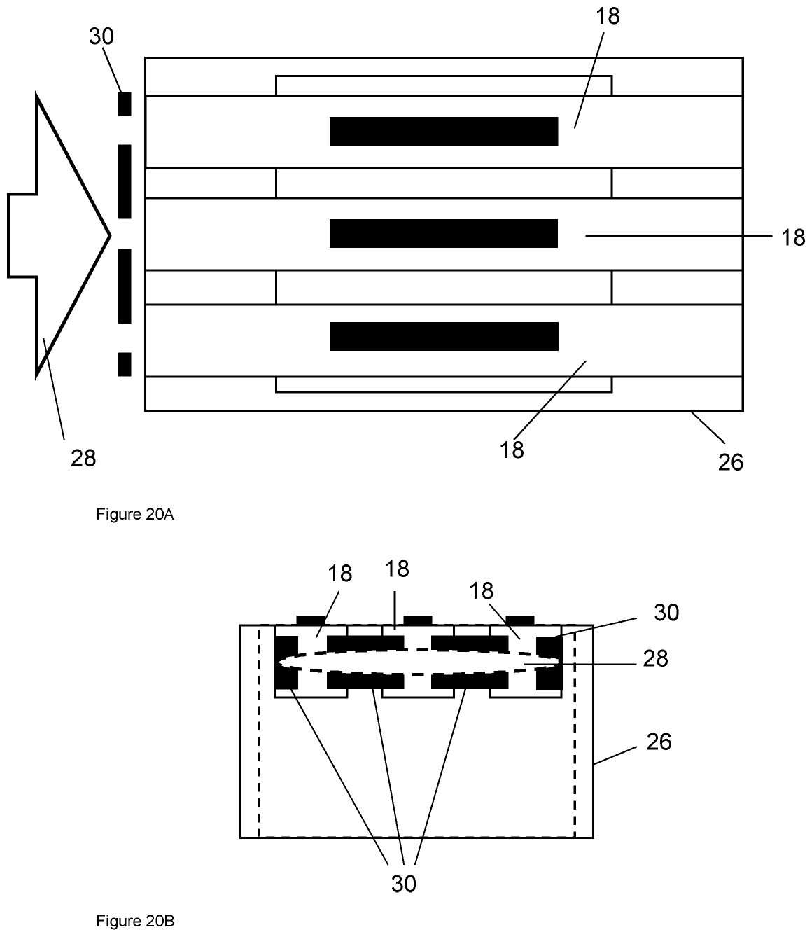

FIG. 20A shows a top view of the chip with multiple optical elements; and

FIG. 20B shows a front view of the chip according to FIG. 20A.

Referring to FIGS. 1 to 7, there is provided an optical element 10 comprising a light pathway 12 comprising an input surface 14, a reflective surface 16 and an output surface 18. The input surface 14 enables light 12, such as an incident light beam, to enter into the optical element 10. The input surface 14 and/or the output surface 18 can be a refractive or a diffractive or a transmissive surface. In some instances, the incident light beam is refracted at the input surface 14 upon entry into the optical element 10. The light is directed towards the reflective surface 16 where one or more capture components 22 such as antibodies are deposited onto the reflective surface 16. The capture components are directly printed onto the reflective surface 16 of the optical element 10, as shown in FIG. 1.

Referring to FIGS. 1 to 7, the reflective surface 16 is able to reflect the light 12 by total internal reflection. As the light reaches the reflective surface 16, the light can be configured to excite the capture components 22. This may cause the capture components 22 to emit light at a specific wavelength. The light may undergo a single reflection or it may undergo multiple reflections at the reflective surface 16. As shown in FIGS. 1 to 7, there is provided a transmission surface 20 which is configured to enable emissions from the capture components 22 to exit the element 10. The transmission surface 20 can be a diffractive, or a refractive or a non-planar surface. The emissions from the capture components may be luminescence for example, fluorescence or phosphorescence. The reflected light 12 can exit the optical element 10 through the output surface 18.

Referring to FIGS. 1 to 3, the optical element 10 may be in a prism form, or a dove prism or a cuboid or any other suitable configuration to enable light to enter, reflect and exit. The optical element can be made from plastic, polymer or glass or any other suitable materials.

As shown in FIGS. 1 and 2, the prism- or cuboid-optical element 10 introduces an incident light beam 12 for a single reflection at the reflective surface 16, where the target components 22 are deposited, through an angular change resulting from refraction. Alternatively, the prism-optical element 10 may introduce an incident light beam 12 for a single reflection at the reflective surface 16 without an angular change resulting from refraction, as shown in FIG. 3.

Referring to FIG. 4, there is provided at least one diffraction grating 28 at the input surface 14 or at the output surface 18 or both. Depending on the location and required path of the light, the diffraction grating 28 may work in transmission or reflection. The diffraction grating 28 may be configured to diffract the light into several beams travelling in different directions. As an example, the diffraction grating 28 located at the input surface 14 may diffract the incident light beam as it enters into the optical element 10. In some embodiments, the diffraction grating can be used to diffract the incident light beam onto the reflected surface 16. In another example, the diffraction grating 28 can diffract the reflected light at the output surface 18. As shown in FIG. 4, the optical element 10, which may be a cuboid optical element, introduces an incident light beam 12 for a single reflection at the reflective surface 16 through an angular change resulting from a diffraction grating 28 working in transmission. Referring to FIG. 5, there is provided at least one diffraction grating 28 located at the transmission surface 20.

Referring to FIG. 6, there is shown an optical element 10 which introduces an incident light beam for a single reflection at the reflective surface 16 through an angular change resulting from at least one diffraction grating 28 working in reflection. The diffraction grating 28 is provided in order to reflect the light within the optical element 10. In some embodiments, the diffraction grating may be configured to provide total internal reflection of the light. The diffraction grating 28 can be positioned at the output surface 18 as shown in FIG. 6. Additionally or alternatively, the diffraction grating may be provided at the input surface and/or at the transmission surface of the optical element.

In an alternative embodiment, not shown in the accompanying drawings, the light beam takes a similar path to that illustrated in FIG. 6, but the reflection comes not from a grating, but from a reflective surface provided by a reflective material being applied to the surface of the optical element. In some examples, this is a silvered surface.

The input surface 14 may have a number of different functionalities as illustrated in FIG. 6. In some instances, the input surface 14 may be configured to allow an incident light beam to enter 24 into the optical element 10 as well as enabling the incident light beam to exit 26 from it. Additionally or alternatively, the output surface 18 may be configured to allow an incident light beam to exit the optical element as well as enabling the incident light beam to enter the optical element. In some embodiments, not illustrated in the accompanying drawings, wherein the optical element is triangular in cross section, the transmission surface 20 may act as an input surface and/or an output surface of the optical element. For example, the transmission surface 20 may be configured to enable light to enter and/or exit the optical element.

Referring to FIG. 7, there is provided an optical element 10 which introduces an incident light beam for a single reflection at the reflective surface 16 through an angular change resulting from an optical lens structure i.e. a Fresnel lens structure 32 located at the transmission surface 20. The incident light beam enters 24 at the transmission surface 20 and through the Fresnel lens structure 32, where the light is directed towards the reflective surface 16. The light then undergoes total internal reflection at the reflective surface 16. The incident light beam is able to exit 26 at the transmission surface 20 through the Fresnel lens structure 32. Moreover, the Fresnel structure 32 could be fabricated more readily with an optical element made from a polymer than with an optical element made from glass.

Referring to FIG. 8, there is provided an optical element 10 to enable light to enter 24 and exit 26. The optical element 10 comprises a reflective surface 16 where the target components 22 are deposited. FIG. 8 shows a waveguiding scenario.

As shown in FIG. 9, there is shown an optical element 10 with a non-planar transmission surface 20. The non-planar surface 20 can be utilised to aid with light collection or imaging applications. This may be particularly useful for optical elements constructed from polymer, since complex volumes can be fabricated with much greater ease and lower cost compared with optical elements made from glass.

The optical element as shown in FIGS. 1 to 9 can form part of an assay cartridge for detecting a target component in a biological fluid such as a saliva or urine or whole blood, plasma or serum sample. The capture component deposited on the reflective surface of the optical element may be selected to capture the target component to be detected. Additionally, the optical element may be disposable that is intended to be used once or a few times.

In some embodiments, the reflective surface of the optical element may be configured to form a portion of a microfluidic flow channel. The target component, which may be in a liquid form, can be deposited directly via printing directly onto the reflective surface of the optical element. In some embodiments, the capture components can be premixed with detection reagents prior to being directly deposited onto the reflective surface. In some embodiments, the receptor molecules may be deposited onto the reflective surface. Additionally or alternatively, the reagents and receptor molecules can be deposited onto the reflective surface, or the detection reagents and the receptor molecules can be deposited onto the reflective surface within diffusion distance.

In addition, the reflective surface of the optical element may further comprise one or more solid layers e.g. polymer film or glass plate, ports, wells, channels, chambers, valves, pumps, heaters or electrodes. Such structures may be integrated into the reflective surface of the optical element and/or in one or more material layers on the reflective surface of the optical element.

The optical element and/or the solid layers can be fabricated using moulding e.g. injection moulding, soft lithography or replica moulding, hot embossing, or nanoimprint lithography, 3D printing e.g. stereolithography, photocuring of inkjet-printed droplets, fused deposition modelling, or two-photon polymerisation, micro- or nanofabrication e.g. photolithography or electron beam lithography, anodic aluminium oxidation, laser-cutting, laser ablation, and/or machining.

Any of the solid layers on top of the reflective surface of the optical element may be assembled or laminated using one or any combination of contact pressure, heat, adhesive, and/or surface activation (ultra violet (UV)/ozone/plasma). Materials through which the laser beam passes are preferably of similar refractive index.

For a typical bioassay, the capture components may need to be deposited onto the reflective surface in an evanescent region, on an interface that is part of the optical element or part of a solid layer above the optical element; reagents may be disposed on a wall or porous medium in a fluid path upstream of the receptors. Methods for depositing or dispensing capture components, and/or receptors may include, but is not limiting to, noncontact deposition e.g. inkjet and/or contact deposition e.g. using dip-pen lithography, capillary tubes, spilt pins or ink stamps. Additionally or alternatively, capture components may be deposited or dispensed or printed directly onto untreated surfaces of the optical element. In some embodiments, it may be beneficial or required to prepare surfaces of the optical element through functionalisation such as using silanisation and/or activation e.g. using UV/ozone.

Additionally or alternatively, functionalisation may be used to immobilise capture components or to passivate the surfaces e.g. reflective surface of the optical element using bovine serum albumin and/or polyethylene glycol, in order to prevent non-specific binding.

Referring to FIG. 10, there is provided an apparatus 40 for detecting the presence and/or the amount of a target component 22 in a biological fluid. The apparatus 40 comprising an assay cartridge including an optical element 10 and a detector 42 for detecting the presence and/or the amount of the emitted light to provide an indication of the presence and/or the amount of the target component 22 within the sample. In addition, there is provided an imaging lens 44, which may be located between the optical element 10 and the detector 42. In some instances, one or more imaging lens may be provided. The imaging lens 44 can be used to focus the emitted light from the target components onto the detector 42, as shown in FIG. 10.

FIG. 11 shows the apparatus 40 with the optical element. Emissions such as fluorescence emissions from the target components 22 exits through the transmission surface of the optical element. A first imaging lens 44 is provided and may be configured to focus the emitted light onto an aperture 46 i.e. a spatial filter. The inclusion of a spatial filter could be utilised to eliminate out-of-plane signals e.g.

out-of-plane fluorescence signals. A second imaging lens 48 is provided which may be configured to focus the remaining emission signal i.e. in-plane fluorescence signal onto the detector 42 e.g. a CCD to form an image of the sample.

FIGS. 12A and 12B demonstrate how the spatial filter 46 can significantly reduce or eliminate out-of-plane fluorescence signals. FIG. 12A shows that the fluorescence signal derived from the target component can be directed by an imaging lens 44 onto the spatial filter (aperture) 46. The light directed towards the spatial filter as shown in FIG. 12A can be referred to as in-plane fluorescence. In this example as illustrated in FIG. 12A, all the fluorescence from the target component is able to pass through the spatial filter 46 to be detected by the detector 42 to provide data of the target component.

As shown in FIG. 12B, the fluorescence signals coming from the optical element 10 are out-of-plane. As a result, the out-of-plane fluorescence contributes to the background and/or noise level and therefore reduces the signal-to-background and/or signal-to-noise ratio. Therefore, the spatial filter 46 as shown in FIG. 12B is configured to eliminate out-of-plane fluorescence and consequently only a fraction passes through the spatial filter (aperture) 46. Eliminating the out-of-plane fluorescence may result in an improved signal-to-background and/or signal-to-noise ratios.

### EXAMPLE 1

### Prism-Based Optical Element

FIG. 13 shows the measured signal-to-background ratio measured for four cartridges on both an exemplary end-launched (non-prismatic optical element) and a prism-based optical element system. As shown in FIG. 13, a prism-based optical element system resulted in a higher signal-to-background ratio, leading to a more sensitive configuration.

This data compares a specific end-launched configuration that deployed a cost efficient substrate that was readily available. It is not possible to generalise the conclusions from this data set to all end-launched configurations. In this specific experiment, the signal-to-background ratio is higher in the prism-based configuration by a factor of between 20 and 40. Without being bound to a specific theory, it is believed that the prism configuration performs better because when the incident light beam undergoes total internal reflection at a boundary between two different optical media, an evanescent field is generated in the lower refractive index medium, exciting fluorophores in close-proximity i.e. within a few hundred nanometres of the surface. However, a fraction of light is also scattered at the reflective surface, therefore contaminating the pure evanescent field, penetrating through the sample solution resulting in background luminescence from luminophores in solution.

Another source of background is autofluorescence from the optical element that the incident light travels through. It is useful to consider the fraction of light intensity that contributes to the desired evanescent field compared to the fraction of light intensity which generates unwanted autofluorescence. This is defined as the evanescence-autofluorescence ratio. The prism optical element is capable of generating an evanescent field with little or no scattered light contamination, owing to the fact that only a single reflection is undergone at a high-quality reflective surface, compared to waveguiding where in effect multiple reflections occur, with each one generating a scattered component. Additionally, during waveguiding a fraction of the light energy can scatter to higher angles such that they are no longer guided by waveguide and are outside the critical angle. This unguided light will travel straight through the sample solution and will thus contribute to the background. Furthermore, due the optical confinement of the waveguiding configuration, the prism optical element configuration has an inherently higher evanescence-autofluorescence ratio. The data presented in FIG. 13 are based on a prism interfaced with a microscope slide using index-matching material.

### EXAMPLE 2

### Single Use Optical Element

FIG. 14 shows a comparison between two testing cartridges based on microscope slides prepared in the same manner. One cartridge can be removed and replaced between each measurement, whilst the other can be kept directly on the reflective surface of the prism optical element for all measurements. It is reasonably expected that the fluorescence signal would decay with increasing numbers of measurement due to photobleaching of the fluorophores. It is shown in FIG. 14 that the signal from the cartridge that was kept on the prism decayed in a smooth fashion, whilst the signal from the cartridge that was removed and replaced between each measurement decayed in a less consistent manner.

A possible route to alleviate this issue can be to develop a deformable polymer layer on the prism to provide index-matching. The microscope slide component of the cartridge could be pressed into the polymer, which would deform to provide optical contact. However, developing such a polymer with all the required properties i.e. refractive index, optical quality, elastic properties and low autofluorescence, is a considerable task and still suffers from some of the issues that plague index-matching fluids, i.e. cleanliness, contaminants and would additionally come with a lifetime which may prove to be problematic. An alternative route is to provide the prism optical element (or similar optical element) as the consumable single-use optical element. This would negate the issues with index-matching materials since they would no longer be required.

Referring to FIG. 15, there is provided a cartridge 10 comprising a sample management module 11 for collecting a fluid sample. The sample is a liquid sample such as a saliva sample.

The sample management module 11 further comprises a lid 22. The lid is provided with a clip 23. The clip 23 is opening resistant such that, under normal conditions, the user is not easily able to re-open the lid once it has been closed. The lid 22 can be closed via action by the user or by any other means.

Referring to FIG. 16, there is provided a preform 100 within a heating chamber 102. The preform 100 can be drawn, typically in a downward direction 104 into an elongate strand 116. The drawing of the preform 10 in a downward direction 114 can be influenced by gravity, or by the use of an actuator. In some instances, pressure may also be applied to draw the preform in a downward direction. The elongate strand 116 can be divided into a plurality of optical elements 118. As shown in FIG. 16, dividing the elongate strand 116 may involve cleaving the strand using a cleaving apparatus or it may involve processing which can be carried out by a laser 120, which may be a CO₂ laser.

The preform 100 may be made from any material such as glass or polymer. In some instances, fused silica is a desirable material for the preform since it exhibits low auto-fluorescence properties.

The preform 100 can be held in the heating chamber 112 such as a furnace on a drawing tower, which is typically several meters tall. The preform 110 is heated by the furnace to a temperature equal to or exceeding the transition temperature of the preform. For example, silica has a transition temperature around 2000° C., whereas some polymers have transition temperatures around 300° C. The preform can be heated to a temperature at or slightly above the transition temperature, but below the crystallisation temperature, as this will facilitate the drawing of the preform 110 into an elongate strand 116.

The elongate strand 116 may then be attached to a rotating drum or caning machine at the bottom of the tower (not shown in the drawings), which can be set to pull the elongate strand down at a controlled and specified rate. Control of the pulling and feeding rates may give accurate control over the dimensions of the elongate strand. The elongate strand can then be spooled onto the drum and transferred to a bobbing for storage. Alternatively, the elongate strand be cut at appropriate lengths and stored in a long glass capillary tube.

Referring to FIGS. 17A and 17B, there is provided a preform 100 and an elongate strand 116. As shown in FIG. 17A, the preform may have a larger side length than the elongate strand. Referring to FIG. 17A, the preform has a square cross section and the side length of the preform may be between 50 mm to 100 mm, or it may be more than 10, 20, 30, 40, 50, 60, 70, 80 or 90 mm. In some embodiments, the side length of the preform may be less than 100, 90, 80, 70, 60, 50, 40, 30 or 20 mm. For example, the depth and/or width of the preform may be 50 mm. A rectangular cross section preform may also be utilised with each side falling within the ranges exemplified above for a square cross section preform.

In some embodiments, the height of the preform can be 100 to 1000 mm, or it may be more than 100, 200, 300, 400, 500, 600, 700, 800 or 900 mm. In some embodiments, the height of the preform may be less than 1000, 900, 800, 700, 600, 500, 400, 300 or 200 mm. For example, the height of the preform is 500 mm.

The preform can be of any shape such as a square, circle or rectangle. The cross section dimension of the preform may be in any suitable dimension that can then be drawn into an elongate strand. As an example, the dimensions of the preform may be 50 mm×50 mm×500 mm.

Referring to FIG. 17B, the cross sectional dimension of the elongate strand may be in any suitable dimension. As an example, the cross sectional dimension of the elongate strand may be 1 mm×1 mm×1.25 km. In another example, the cross section of the elongate strand may be more than 1 mm×1 mm, 2 mm×2 mm, 4 mm×4 mm, 6 mm×6 mm or 8 mm×8 mm. In some embodiments, the cross section dimension of the strand may be less than 10 mm×10 mm, 8 mm×8 mm, 6 mm×6 mm, 4 mm×4 mm or 2 mm×2 mm.

The elongate strand can then be processed into individual optical elements. This can be achieved by utilising a mechanical cleaver, which is essentially controlled breaking of the elongate strand to produce an optical surface. For example, commercially available mechanical cleavers can be used to cleave the elongate strand at an angle of up to 15° to create an optical element e.g. a prism optical element such as a micro-dove prism. In some embodiments, the optical element (not shown in the accompanied drawings) may be a cuboid, rectangular or a triangular optical element. Moreover, the elongate strand can be cleaved by the mechanical cleaver at any angle to create a plurality of optical element.

Additionally or alternatively, CO₂ or other laser processing can be used to process the elongate strand into a plurality of optical elements. Using a laser to divide the elongate strand can produce excellent surface finish which may not require further polishing when used in the context of a component of an assay cartridge.

The fabrication process may also include the step of polishing and/or cleaning the surfaces of the preform, elongate strand and/or the optical element.

Although the illustrated embodiment shows a rectilinear preform and corresponding elongate strand, other geometries are possible without departing from the methodology described. For example, a triangular or octagonal preform can be drawn and then cut orthogonally to create an optical element that is triangular or octagonal in cross section.

As shown in FIGS. 18A and 18B, there is provided, respectively, a side view and top view of an optical element 118. The optical element 118 can receive an incident light beam 122 and the incident light beam can be reflected via total internal reflection at the evanescent region 124. The length of the optical element may be between 1 mm to 30 mm, or it may be more than 1, 5, 10, 15, 20 or 25 mm. In some embodiments, the length of the optical element may be less than 30, 25, 20, 15, 10 or 5 mm. In some embodiments, the length of the optical element is between 10 and 20 mm. In some embodiments, the length of the optical element is approximately 13 mm.

The beam width that enters or exits the optical element may be between 0.1 to 2 mm, or it may be more than 0.1, 0.5, 1 or 1.5 mm. In some embodiments, the beam width may be less than 2, 1.5, 1 or 0.5 mm. For example, the beam width is less than or equal to 0.5 mm.

Depending on the shape of the preform and the angle of the cleaving, the optical element may have an evanescent region, adjacent to a reflection surface that is either a cleaved surface, in the case of a triangular or octagonal cross section preform; or a drawn surface, in the case of a rectilinear cross section preform.

Referring to FIGS. 19A and 19B, there is shown a top view (FIG. 19A) and a front view (FIG. 19B) of an optical element 118 being interfaced with a test cartridge/chip 126. An input light beam 128 can enter the optical element on the chip interface. The chip interface can be fabricated from injection moulded polymer.

Referring to FIGS. 20A and 20B, there is provided a top view (FIG. 20A) and a front view (FIG. 20B) of the chip interface 126. As shown in FIGS. 20A and 20B, a plurality of optical elements 118 can be interfaced in a single chip 126, therefore maximising the interrogation area. The number of optical elements 118 that can interface in a chip 126 can vary substantially. An aperture or a mask 130 can also be used to spatially shape the input collimated laser beam in order to illuminate the desired target regions.

Various further aspects and embodiments of the present invention will be apparent to those skilled in the art in view of the present disclosure.

“and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

Unless context dictates otherwise, the descriptions and definitions of the features set out above are not limited to any particular aspect or embodiment of the invention and apply equally to all aspects and embodiments which are described.

It will further be appreciated by those skilled in the art that although the invention has been described by way of example with reference to several embodiments. It is not limited to the disclosed embodiments and that alternative embodiments could be constructed without departing from the scope of the invention as defined in the appended claims.

1. An assay cartridge for detecting a target component in a fluid, the assay cartridge including an optical element comprising:
a light pathway comprising an input surface, reflective surface and output surface configured to enable light to enter, reflect and create an evanescent field in the vicinity of the reflective surface and exit the element;
a plurality of capture components deposited on the reflective surface in the vicinity of the evanescent field; and
a transmission surface configured to enable emissions from the evanescent field to exit the element;
wherein the assay cartridge is a single use cartridge.

2. The assay cartridge according to claim 1, wherein the single use nature of the cartridge is implemented through physical constraints.

3. The assay cartridge according to claim 2, further comprising a one way clip to ensure that the cartridge is single use.

4. The assay cartridge according to claim 1, wherein the single use nature of the cartridge is implemented through chemical constraints.

5. The assay cartridge according to claim 4, further comprising an irreversible spot to ensure that the cartridge is single use.

6. The assay cartridge according to claim 1, wherein the single use nature of the cartridge is implemented through data management.

7. The assay cartridge according to claim 6, further comprising an identity tag to ensure that the cartridge is single use.

8. The assay cartridge according to claim 7, wherein the identity tag is printed onto the cartridge.

9. The assay cartridge according to claim 7 or claim 8, wherein the identity tag is selected from a group including a barcode or a QR code.

10. The assay cartridge according to claim 7, wherein the identity tag is an RFID tag.

11. The assay cartridge according to any one of claims 1 to 10, wherein the emissions from the evanescent field are Mie, Raman or Rayleigh scattering.

12. The assay cartridge according to any one of claims 1 to 11, wherein at least one of the capture components is DNA or an antibody or a protein.

13. The assay cartridge according to any one of claims 1 to 12, wherein the fluid is saliva.

14. A method of fabricating an array of assay cartridges each cartridge being an assay cartridge according to any one of claims 1 to 13, the method comprising the steps of:
fabricating a plurality of optical elements through the steps of:
heating a preform to a temperature equal to or exceeding the glass transition temperature of the preform;
drawing the preform into an elongate strand; and
dividing the strand into a plurality of optical elements;
mounting at least one optical element in each assay cartridge; and
depositing at least one capture component on the reflective surface of each optical element.

15. The method according to claim 14, wherein the dividing of the strand into a plurality of optical elements involves cleaving the strand.

16. The method according to claim 14, wherein the dividing of the strand into a plurality of optical elements involves processing the strand with a laser.

17. The method according to claim 14, 15 or 16, wherein the dividing of the strand occurs perpendicular to the direction in which the preform is drawn.

18. The method according to claim 15, wherein the cleaving step takes place alternately from each side of the strand so that each optical element has a trapezoidal cross section.

19. The method according to any one of claims 14 to 18, wherein the assay cartridge includes a plurality of optical elements.

20. The method according to any one of claims 14 to 19, wherein the depositing of the capture component occurs by printing.
